(19) United States
(12) Patent Application Publication
(10) Pub. No.: US 2026/0175016 A1
(43) Pub. Date: Jun. 25, 2026

(54) MICROELECTRICAL STIMULATION SYSTEM FOR REGULATING UTERINE CONTRACTION AND CONTROL METHOD THEREOF

(71) Applicant: 4P LAB CO., LTD., Seoul (KR)

(72) Inventors: Ki Hoon AHN, Seoul (KR); Jay KIM, Seoul (KR); Eun Jin WANG, Seoul (KR)

(73) Assignee: 4P LAB CO., LTD., Seoul (KR)

(21) Appl. No.: 19/128,893

(22) PCT Filed: Oct. 17, 2023
(86) PCT No.: PCT/KR2023/016070

§ 371 Date: May 9, 2025

(30) Foreign Application Priority Data
Nov. 10, 2022 (KR) 10-2022-0149319

(51) Int. Cl. A61N 1/05; A61B 5/00; A61N 1/08; A61N 1/36; A61N 1/372

(52) U.S. Cl. CPC A61N 1/0534; A61B 5/6882; A61N 1/08; A61N 1/36007; A61N 1/36017; A61N 1/36031; A61N 1/36034; A61N 1/36071; A61N 1/37235; A61N 1/37247

(57) ABSTRACT

The present invention relates to a micro-electrical stimulation system for regulating uterine contraction and control method thereof. According to the micro-electrical stimulation system includes a body that is non-invasively inserted into the uterus; a stimulation neural electrode that inhibits uterine contraction by applying micro-electrical stimulation to the uterine inlet nerve; and a controller that sets the intensity, interval, and duration of the micro-electrical stimulation, wherein the controller sets the micro-electrical stimulation within an intensity of 100 to 5000 μA, an interval of 100 to 200 μsec, and a duration of 3 to 20 seconds.

### TECHNICAL FIELD

The present invention relates to a micro-electrical stimulation system for diagnosing or inhibiting preterm labor in pregnant women by applying micro-electrical stimulation through a non-invasive neural electrode assembly.

### BACKGROUND ART

In obstetrics and gynecology, the fetal heart rate is generally detected using the principles of ultrasound, and changes in the heartbeat are used to examine the health and abnormal signs of the fetus. In other words, pregnant women could regularly visit the hospital for ultrasound examinations to check the condition of the fetus.

However, recently, due to changes in social environment such as stress from excessive work environments related to social activities and environmental pollution, there has been an increasing trend of preterm births, where babies are born earlier than the expected delivery date. Accordingly, in addition to examinations to measure the fetal heart rate, examinations to measure uterine contraction levels of pregnant women are also performed to examine the risk of preterm labor.

The main examination and diagnostic methods related to preterm labor include checking changes in the cervix through internal examination, confirming amniotic fluid leakage through speculum examination, and confirming chorioamnionitis through amniocentesis. Currently, drug therapies using uterine contraction inhibitors (ritodrine, atosiban, magnesium, etc.) and antibiotics have been mainly implemented in relation to preterm labor.

However, conventional examination methods for predicting preterm labor are often questioned for their diagnostic utility due to low accuracy, and drug treatments used in medical settings for the prevention or treatment of preterm labor also have limited effectiveness.

Meanwhile, the prior art document presented below introduces the induction mechanism of preterm labor, various causes involved, and treatment strategies, but it is still insufficient to suggest clear prevention and treatment methods for preterm labor.

“Recent Treatment Insights on Preterm Labor”, Kim Jong-hwa, Korean Society of Obstetrics and Gynecology, 2005.

### DISCLOSURE

### Technical Problem

The purpose of the present invention is to provide a micro-electrical stimulation system for uterine contraction control and its control method that diagnoses or inhibits preterm labor in pregnant women by applying micro-electrical stimulation through a non-invasive neural electrode assembly.

### Technical Solution

To solve the above problem, the present invention relates to a micro-electrical stimulation system for uterine contraction control and its control method. According to the first aspect of the present invention, the micro-electrical stimulation system for uterine contraction control includes a body that is non-invasively inserted into the uterus; a stimulation neural electrode that inhibits uterine contraction by applying micro-electrical stimulation to the uterine inlet nerve; and a controller that sets the intensity, interval, and duration of the micro-electrical stimulation, wherein the controller sets the micro-electrical stimulation within an intensity of 100 to 5000 μA, an interval of 100 to 200 μsec, and a duration of 3 to 20 seconds.

According to an embodiment of the present invention, the system further includes a recording neural electrode that measures neural signal information according to uterine contraction; and a database unit that receives and stores the neural signal information from the recording neural electrode, wherein the controller selectively forms a uterine contraction delay and inhibition state by applying at least two different micro-electrical stimulations based on the stored neural signal information.

According to an embodiment of the present invention, the controller acquires individual uterine contraction delay and inhibition information through changes in the neural signal information according to the intensity of the applied micro-electrical stimulation, and determines the intensity, interval, and duration values of each micro-electrical stimulation based on the individual uterine contraction delay and inhibition information.

According to the second aspect of the present invention, the control method for the micro-electrical stimulation system includes: (a) setting the micro-electrical stimulation within an intensity of 100 to 5000 μA, an interval of 100 to 200 μsec, and a duration of 3 to 20 seconds; and (b) applying the set micro-electrical stimulation to the uterine inlet nerve by a stimulation neural electrode.

According to an embodiment of the present invention, after step (b), the method further includes: (c) measuring neural signal information according to uterine contraction by a recording neural electrode; (d) receiving and storing the neural signal information from the recording neural electrode; and (e) selectively forming a uterine contraction delay and inhibition state by applying at least two different micro-electrical stimulations based on the stored neural signal information.

According to an embodiment of the present invention, step (e) includes: (e-1) acquiring individual uterine contraction delay and inhibition information through changes in the neural signal information according to the intensity of the applied micro-electrical stimulation; and (e-2) determining the intensity, interval, and duration values of each micro-electrical stimulation based on the individual uterine contraction delay and inhibition information.

### Advantageous Effects

According to the present invention, the physiological phenomenon of uterine muscle contraction that appears in the induction mechanism of preterm labor can be detected to predict or continuously monitor the signs of preterm labor.

In addition, when uterine contraction is anticipated based on the detected neural signals, the uterine muscle contraction can be inhibited or delayed by automatically applying electrical stimulation to the uterine inlet nerve using a non-invasive neural electrode, thereby detecting preterm labor in advance.

### DESCRIPTION OF DRAWINGS

FIG. 1 is a graph illustrating the trend of infant mortality rate according to gestational age.

FIG. 2 is an exploded perspective view of a non-invasive neural electrode assembly according to the present invention.

FIG. 3 is a front view of the neural electrode assembly of FIG. 2 according to an embodiment of the present invention.

FIG. 4 is a diagram explaining the operation method of a non-invasive neural electrode control system according to the present invention.

FIG. 5 is a schematic block diagram of the neural electrode control system of FIG. 4.

FIG. 6 is a flowchart showing each step of a micro-electrical stimulation control method for uterine contraction control according to the present invention.

FIG. 7 is a diagram and graph showing the uterine contraction inhibitory effect using neural electrodes in a mid-sized animal (pig) pregnant sow.

FIG. 8 is a graph showing the decrease in uterine contraction after micro-electrical stimulation in normal pregnant mice and preterm labor mice.

FIG. 9 is a graph confirming uterine contraction in maternal uterine smooth muscle after potassium chloride (KCl) and oxytocin pretreatment (incubation).

FIGS. 10 (a) and (b) are graphs showing uterine contraction delay and inhibition according to the intensity, interval, and duration of micro-electrical stimulation applied to human uterine smooth muscle.

FIGS. 11 (a) and (b) are graphs showing uterine contraction delay and inhibition according to the intensity, interval, and duration of micro-electrical stimulation.

FIG. 12 is a graph confirming that uterine contraction is reduced at a certain range of micro-electrical stimulation intensity for a total of 13 mothers.

### DESCRIPTION OF REFERENCE NUMERALS

10: Neural electrode assembly
11: Cover
12: Recording neural electrode
13: Stimulation neural electrode
15: Processor
17: Communication unit
19: Power supply
20: Monitoring terminal
200: Controller
300: Database unit

### MODE FOR DISCLOSURE

Hereinafter, embodiments of the present specification will be described in detail with reference to the drawings. However, detailed descriptions of well-known functions or configurations that may obscure the gist of the embodiments are omitted from the following description and the attached drawings. In addition, throughout the specification, it should be understood that when a component “includes” something, it does not exclude other components unless specifically stated otherwise, but may include other components.

The terms used in this specification are used only to explain specific embodiments and are not intended to limit the specification. The singular expressions include plural expressions unless the context clearly indicates otherwise. In this application, the terms “include” or “have” and the like are used to specify the presence of stated features, numbers, steps, operations, components, parts, or combinations thereof, and do not preclude the presence or addition of one or more other features, numbers, steps, operations, components, parts, or combinations thereof.

Unless defined otherwise, all technical and scientific terms used herein, including all terms generally understood by those skilled in the art to which this specification belongs, have the same meaning. Generally used dictionary definitions should be interpreted as having meanings consistent with the context of relevant technology and are not to be interpreted in an idealized or overly formal sense unless expressly defined in this specification.

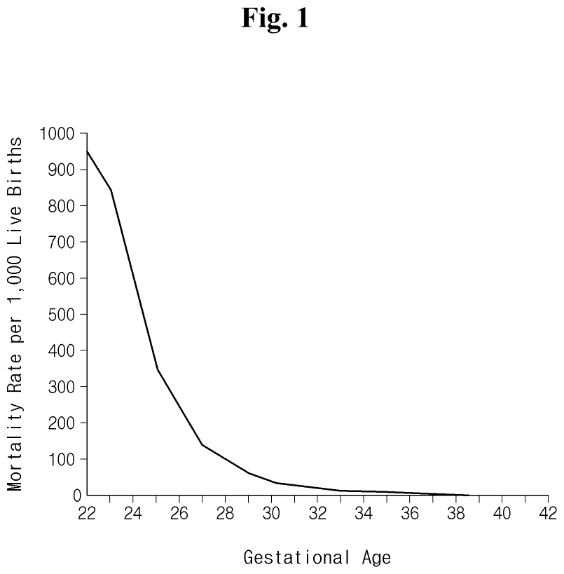

Referring to FIG. 1, which is a graph illustrating the trend of infant mortality rate according to gestational age, it can be seen that the shorter the gestational age, the extremely higher the infant mortality rate, while the mortality rate drops dramatically as the risk of preterm labor is overcome and approaches normal delivery date. Currently, preterm labor occurs in about 10% of pregnancies and has been identified as the most important cause of neonatal mortality, affecting low birth rates and population decline. In particular, along with this infant mortality rate trend, statistics showing an annual increase in the incidence of preterm or low birth weight infants also support the need for preterm labor risk management.

Infection is known to be the most important factor in the causes of preterm labor, and other causes include vascular abnormalities, decidual aging, uterine overdistension, decreased progesterone action, cervical disease, breakdown of maternal-fetal immunological tolerance, and stress. Although various causes are involved in the induction mechanism of preterm labor, the final physiological phenomena of uterine muscle contraction and cervical dilation are common manifestations.

Currently, examinations such as ultrasonic measurement of cervical length, measurement of fatal fibronectin in vaginal fluid, and measurement of MMP-8 concentration in amniotic fluid are being conducted to predict preterm labor. However, the accuracy is not high, raising questions about their diagnostic utility. In addition, not only the prediction of preterm labor but also its prevention and treatment are still far from adequate, and the therapeutic effects of progesterone, antibiotics, and tocolytics that are widely used clinically are also minimal in reality.

Meanwhile, under the above-mentioned problem awareness, attention was paid to the fact that the organs located in the pelvis are under rich autonomic nervous system regulation. In the pelvis, there are the uterus, bladder, rectum, and anus, and all these organs are regulated for muscle contraction and relaxation functions under the control of the autonomic nervous system, including the sympathetic and parasympathetic nerves. In particular, stimulation of the parasympathetic nerves causes contraction of pelvic organs, while stimulation of the sympathetic nerves causes relaxation of pelvic organs. Therefore, based on research indicating that pelvic organs, including the uterus, receive considerable neurological regulation, it was expected that the spontaneous preterm labor could ultimately be treated by regulating neural transmission in the pelvis.

Based on this technical understanding, this specification proposes to identify the nerves involved in uterine muscle contraction or cervical dilation among the pelvic organs, and to provide technical means that can effectively control uterine muscle contraction by stimulating the sympathetic nerves through these nerves. In addition, a non-invasive neural electrode that is inserted non-invasively adjacent to the uterine inlet nerve and has excellent biocompatibility was introduced to minimize patient discomfort, and a control model was established to measure uterine contraction or stimulate the uterine inlet nerve through various types of neural electrodes. In particular, these technical means were integrated into a single miniaturized device to enable early diagnosis and treatment of spontaneous preterm labor.

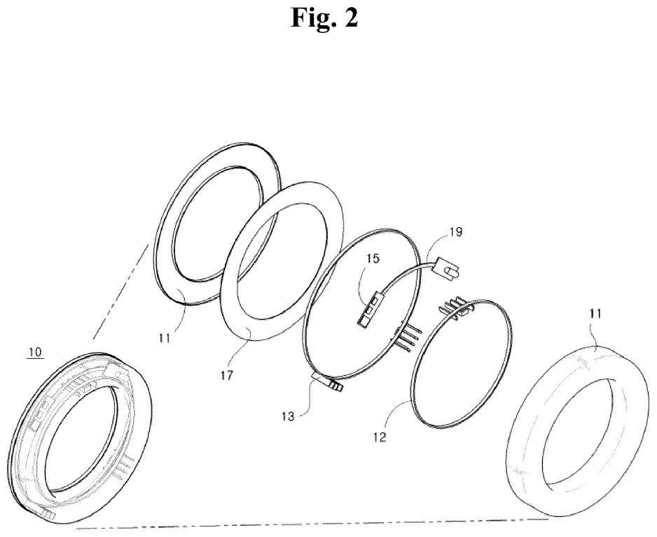

FIG. 2 is an exploded perspective view of a non-invasive neural electrode assembly according to the present invention.

Referring to FIG. 2, the micro-electrical stimulation system for uterine contraction control according to the present invention includes a neural electrode assembly (10), and a controller (200) and a database unit (300) that operate in conjunction with it.

The body of the neural electrode assembly (10) is configured to be non-invasively inserted into the uterus and can be covered with a cover (11) formed of a biocompatible material harmless to the human body or have its surface coated with a biocompatible material. This body or cover (11) can be made of any material that does not react with human tissue. For example, it can be formed of a biocompatible material such as EVA copolymer (Ethylene Vinyl Acetate Copolymer). Alternatively, it can be formed including various materials such as PE (Poly Ethylene), PS (Poly Styrene), PET (Poly Ethylene Terephthalate), PVC (Poly Vinyl Chloride), PVDC (Poly Vinylidene Chloride), PP (Poly Prophylene), PVA (Poly Vinyl Alcohol), which are plastic materials with high transparency and elasticity, thereby exhibiting excellent bonding characteristics.

From a structural perspective, the body can be implemented in a ring shape as exemplified in FIG. 2, but in addition to this shape, it can be formed in various shapes suitable for non-invasive insertion into the uterus, such as a disc shape, a spherical shape, a cylindrical shape, a rugby ball shape, or a banana shape. It is preferable for the body to be structurally fixed to the uterine wall adjacent to the cervix after insertion.

The recording neural electrode (12) is formed to measure neural signal information according to uterine contraction and is coupled to the body. The recording neural electrode (12) continuously monitors neural signals according to uterine contraction by being positioned to wrap around the cervix when the body is inserted into the uterus, thereby predicting and detecting early labor.

The stimulation neural electrode (13) is formed to stimulate the uterine inlet nerve to inhibit uterine contraction and is coupled to the body. The stimulation neural electrode (13) can stimulate the uterine inlet nerve to inhibit uterine contraction when uterine contraction is detected through the recording neural electrode (12). In particular, it is preferable for the stimulation neural electrode (13) to apply neural stimulation signals of an intensity corresponding to the detected degree of uterine contraction to the uterine inlet nerve to prevent or delay preterm labor.

The stimulation neural electrode (13) stimulates the uterine inlet nerve (mainly sympathetic nerve) of the sacral ligament by being positioned adjacent to the sacral ligaments on both sides of the cervix when the body is inserted into the uterus, and can inhibit uterine contraction by applying appropriate electrical stimulation during preterm labor.

In addition to the above configuration, the neural electrode assembly (10) of FIG. 2 includes a communication unit (17) that can transmit the measured signals to a monitoring terminal located outside the body or receive commands to inhibit uterine contraction from the monitoring terminal. Considering the ring-shaped body exemplified in FIG. 2, the communication unit (17) can be arranged in the form of a wireless coil along the inside of the body.

Meanwhile, a power supply (19) and a processor (15) can be further included for driving and controlling the neural electrode assembly (10). The power supply (19) supplies the necessary power to the neural electrode assembly (10), and the processor (15) is electrically connected to the components (12, 13, 17, 19) of the neural electrode assembly (10) to control each or induce command execution.

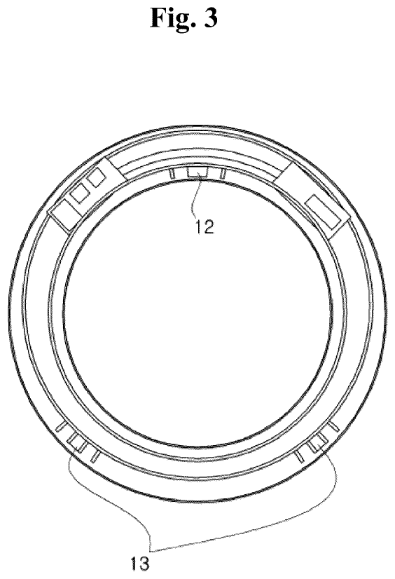

FIG. 3 is a front view of the neural electrode assembly of FIG. 2 according to an embodiment of the present invention, showing that the neural electrode assembly includes two main types of neural electrodes.

Referring to FIG. 3, first, the recording neural electrode (12) is arranged to wrap around the cervix and detects the corresponding bio-signals when uterine muscle contraction occurs.

Second, the stimulation neural electrode (13) can inhibit uterine muscle contraction by stimulating the sympathetic nerves entering the uterus through electrodes positioned in the 4 o'clock and 8 o'clock directions. From an application perspective, if a contraction signal is detected from the uterus through the recording neural electrode (12), it can also quickly inhibit the uterine muscle by automatically stimulating the uterine inlet nerve.

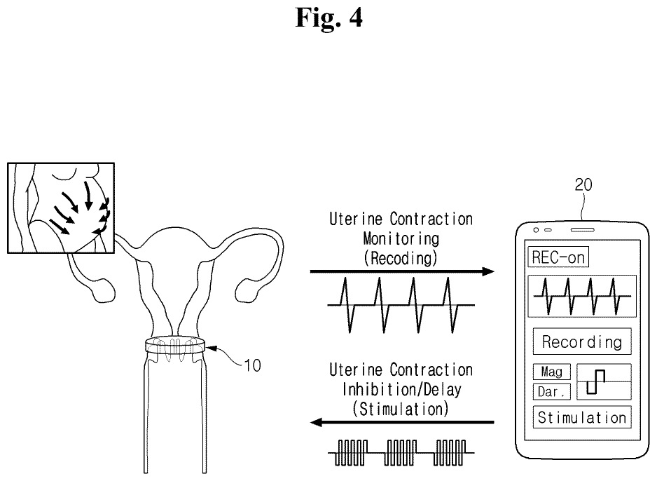

FIG. 4 is a diagram explaining the operation method of a non-invasive neural electrode control system according to the present invention.

Referring to FIG. 4, it is preferable for the neural electrode assembly (10) to be positioned adjacent to the cervix after being inserted non-invasively through the vagina, and it can communicate with a monitoring terminal (20) or monitoring server outside the body by having an integrated wireless communication module. For example, the neural electrode assembly (10) can be worn by high-risk pregnant women for preterm labor at 20-25 weeks of pregnancy, and if necessary, it can also combine inhibition of uterine contraction with drugs by mounting drugs on part of the body.

Now, the monitoring terminal (20), which receives uterine muscle contraction signals measured from the neural electrode assembly (10) positioned at the cervix, can diagnose preterm labor through uterine contraction monitoring. For example, through an application installed on the monitoring terminal (20), the uterine contraction status can be observed and recorded in real-time, and when uterine muscle contraction exceeding the threshold range is detected, a command to inhibit or delay uterine contraction is transmitted to the neural electrode assembly (10). Then, the neural electrode assembly (10) inhibits or delays uterine contraction through electrical stimulation within the human tolerance range according to the command, and as a result, can prevent preterm labor.

Of course, the application of neural stimulation signals for inhibiting uterine contraction can be based on the command of the monitoring terminal (20) as exemplified in FIG. 4, but it can also be directly controlled within the neural electrode assembly (10) depending on the implementation requirements. In this case, the neural electrode assembly (10) judges the normal range based on the uterine contraction signals measured by itself, and directly stimulates the sympathetic nerves to inhibit uterine contraction according to the judgment result.

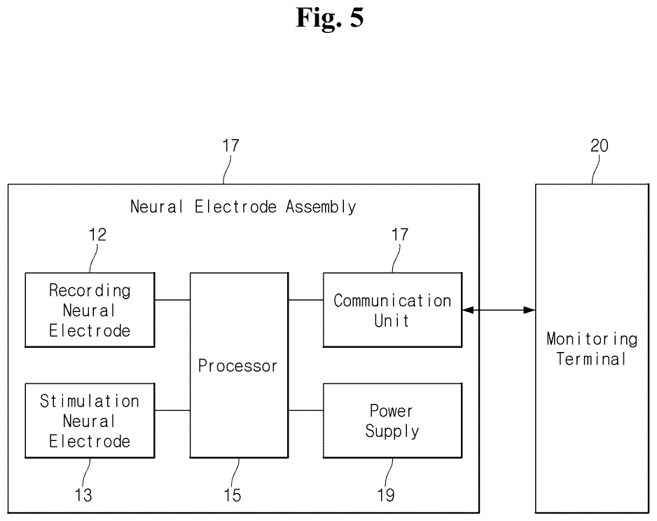

FIG. 5 is a schematic block diagram of the neural electrode control system of FIG. 4, where only an overview of each component is briefly described to avoid repetition of explanation.

The neural electrode assembly (10), which is non-invasively inserted into the uterus, includes a recording neural electrode (12) formed to measure neural signals according to uterine contraction, and a stimulation neural electrode (13) formed to stimulate the uterine inlet nerve to inhibit uterine contraction.

In addition, the neural electrode assembly (10) further includes a communication unit (17) that is electrically connected to the recording neural electrode (12) and the stimulation neural electrode (13) to wirelessly transmit and receive signals and commands. The communication unit (17) transmits neural signals measured through the recording neural electrode (12) to the monitoring terminal (20) and receives commands for uterine contraction inhibition from the monitoring terminal (20) to control the stimulation neural electrode (13). The specifications of the communication unit implemented through the prototype of this neural electrode assembly are exemplified in Table 1 below.

TABLE 1
Wireless Power Transmission System

| Coil Diameter | 26.5 | mm |
|---|---|---|
| Input Power | 19 V / 200 mA | |
| Output Power | 5 V / 510 mA | |
| Frequency | 125 | kHz |
| Max. Distance | 6 | mm |

In addition, the neural electrode assembly (10) includes a power supply (19) that supplies power to each component, and a processor (15) in which algorithms for controlling neural signal recording or electrical stimulation are integrated. The specifications of the power supply (19) and algorithms for controlling neural signal recording or electrical stimulation integrated in the processor (15) implemented through the prototype of this neural electrode assembly are exemplified in Table 2 and Table 3 below.

TABLE 2
Battery

| | |
|---|---|
| Rated Capacity | 300 mAh @ 3.7 V |
| Charging Condition | 250 mA @ 4.2 V |
| Charging Time | 1~1.2 hour |

TABLE 3
Wireless Data Communication System

| Power Consumption | Active: 16 mW | |
|---|---|---|
| | Idle: 3.13 mW | |
| | Sleep: 0.95 uW | |
| Data Rate | Max. 800 | kbps |
| Carrier Frequency | 402~405 | MHz |
| Wake-up Frequency | 2.45 | GHz |

| | |
|---|---|
| Modulation | FSK, bidirectional |

The monitoring terminal (200) communicates with the neural electrode assembly (10) to record, analyze, visualize the measured bio-signals, or transmit control commands to inhibit or delay uterine contraction.

Through the above neural electrode assembly of the present invention, the physiological phenomenon of uterine muscle contraction that appears in the induction mechanism of preterm labor can be detected to predict or continuously monitor the signs of preterm labor, and when uterine contraction is anticipated based on the detected neural signals, the uterine muscle contraction can be inhibited or delayed by automatically applying electrical stimulation to the uterine inlet nerve using a non-invasive neural electrode, thereby detecting preterm labor in advance.

To control the neural electrode assembly (10), the controller (100) is implemented as a device that can be connected to the monitoring terminal (20), the inside of the neural electrode assembly (10), or externally in communication with the neural electrode assembly (10). The controller (100) sets the intensity, interval, and duration of the micro-electrical stimulation applied by the stimulation neural electrode (13) of the neural electrode assembly (10). The controller (100) sets the micro-electrical stimulation within an intensity of 100 to 5000 μA, an interval of 100 to 200 μsec, and a duration of 3 to 20 seconds. Meanwhile, the database unit (200) receives and stores neural signal information from the recording neural electrode (12).

The controller (100) selectively forms a uterine contraction delay and inhibition state by applying two different micro-electrical stimulations based on the neural signal information stored in the database unit (200). The controller (100) acquires individual uterine contraction delay and inhibition information through changes in the neural signal information according to the intensity of the applied micro-electrical stimulation. The controller (100) determines the intensity, interval, and duration values of each micro-electrical stimulation based on the individual uterine contraction delay and inhibition information.

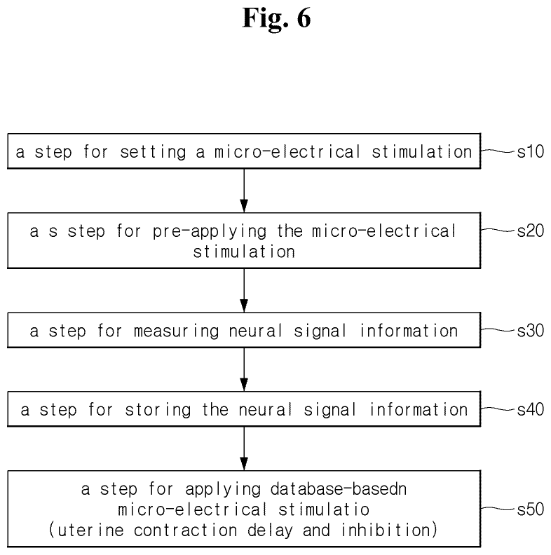

FIG. 6 is a flowchart showing each step of a micro-electrical stimulation control method for uterine contraction control according to the present invention.

Referring to FIG. 6, the micro-electrical stimulation for uterine contraction control according to the present invention is controlled in the following order. The micro-electrical stimulation control method for uterine contraction control according to the present invention includes a step (s10) for setting a micro-electrical stimulation, a s step (s20) for pre-applying the micro-electrical stimulation (s20), a step (s30) for measuring neural signal information, a step (s40) for storing the neural signal information, and a step (s50) for applying database-based micro-electrical stimulation (uterine contraction delay and inhibition).

First, the controller (100) sets the micro-electrical stimulation within an intensity of 100 to 5000 μA, an interval of 100 to 200 μsec, and a duration of 3 to 20 seconds (s10). Next, the stimulation neural electrode (13) applies the set micro-electrical stimulation to the uterine inlet nerve (s20). The recording neural electrode (12) measures neural signal information according to uterine contraction (s30). The database unit (200) receives and stores the measured neural signal information from the recording neural electrode (12) (s40). Next, the controller (100) selectively forms a uterine contraction delay and inhibition state by applying at least two different micro-electrical stimulations based on the stored neural signal information (s50).

The uterine contraction delay and inhibition state formation step (s50) includes an individual uterine contraction delay and inhibition information acquisition step (s51), and a micro-electrical stimulation value determination step (s52). First, individual uterine contraction delay and inhibition information is acquired through changes in the neural signal information according to the intensity of the applied micro-electrical stimulation (s51). Next, the intensity, interval, and duration values of each micro-electrical stimulation are determined based on the individual uterine contraction delay and inhibition information (s52).

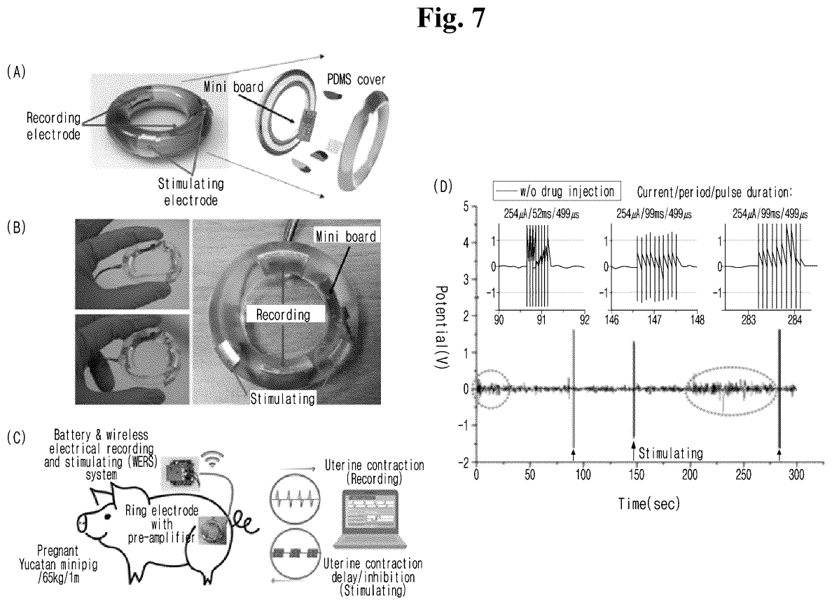

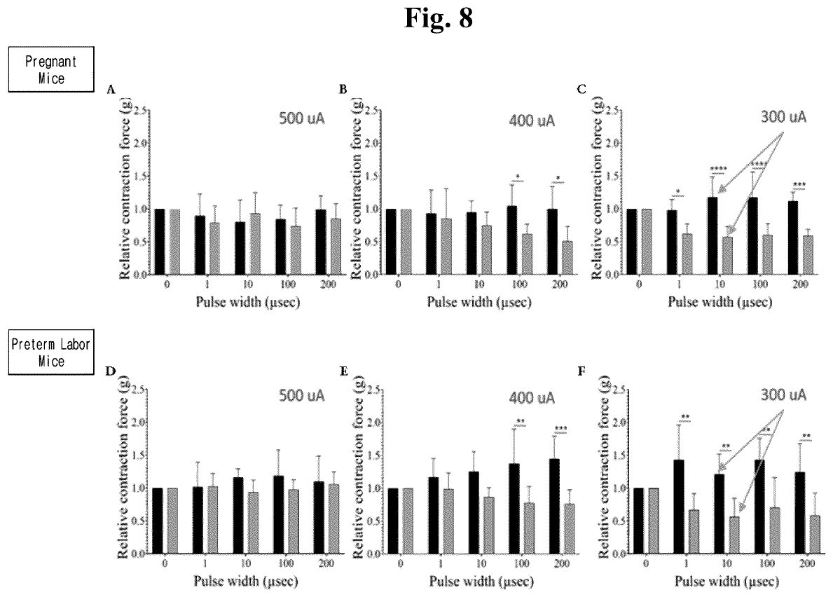

FIG. 7 is a diagram and graph showing the uterine contraction inhibitory effect using neural electrodes in a mid-sized animal (pig) pregnant sow. FIG. 8 is a graph showing the decrease in uterine contraction after micro-electrical stimulation in normal pregnant mice and preterm labor mice.

Referring to FIG. 7 and FIG. 8, using the ring-shaped neural electrode assembly (10) according to the present invention, uterine contraction/relaxation monitoring and uterine contraction inhibition according to electrical stimulation were analyzed in mid-sized animals (mini pigs). When pulse amplitudes of from 500 μA to 300 μA were applied to uterine smooth muscle tissue of normal pregnant mice and preterm labor mice, it was confirmed that uterine contraction decreased. To apply the micro-electrical stimulation protocol to the established ring-shaped neural electrode assembly (10), it is necessary to set the micro-electrical stimulation intensity that controls maternal uterine smooth muscle tissue after micro-electrical stimulation using human maternal smooth muscle tissue.

The process of the myography experiment on maternal uterine smooth muscle tissue is as follows.

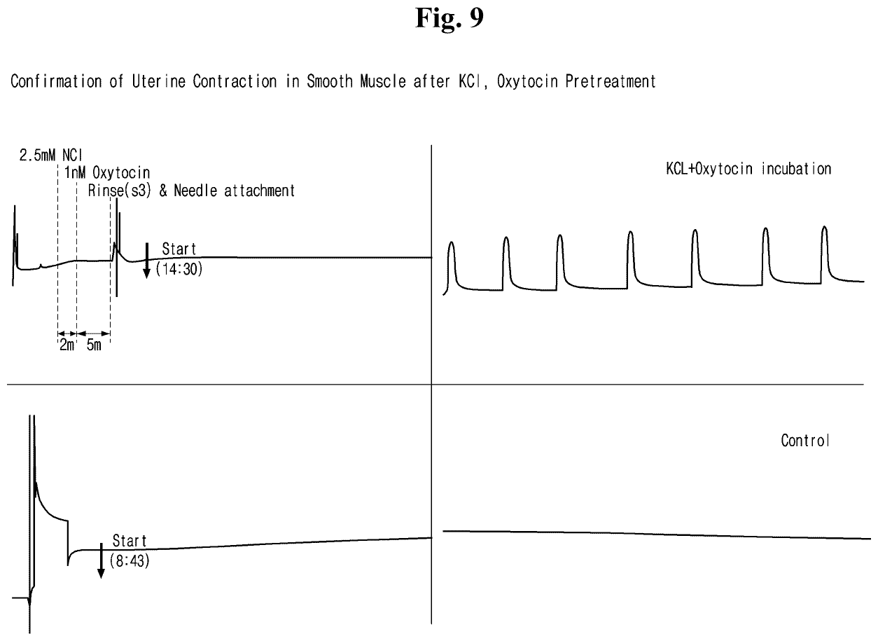

First, the incident site upper part and uterine low segment are incised at 20 mm×30 mm during a cesarean section. Next, the uterine tissue is cut to the same thickness (3 mm×20 mm). This involves cutting 3 layers of tissue and handling it at a length of 5 mm out of the 20 mm length. After handling the tissue, it is hung on the myography device. In the myography device, the tissue length is stretched by 1.5 times from 5 mm to 7.5 mm. KCL 2.5 mM is treated for 2 minutes and Oxytocin 1 nM for 5 minutes in 8 ml of Krebs solution (Krebs-Henseleitsolution). After treatment, washing with Krebs solution is performed 3 times. As a result of the experiment, as shown in FIG. 9, uterine contraction was confirmed in maternal uterine smooth muscle after potassium chloride (KCl) and oxytocin pretreatment (incubation).

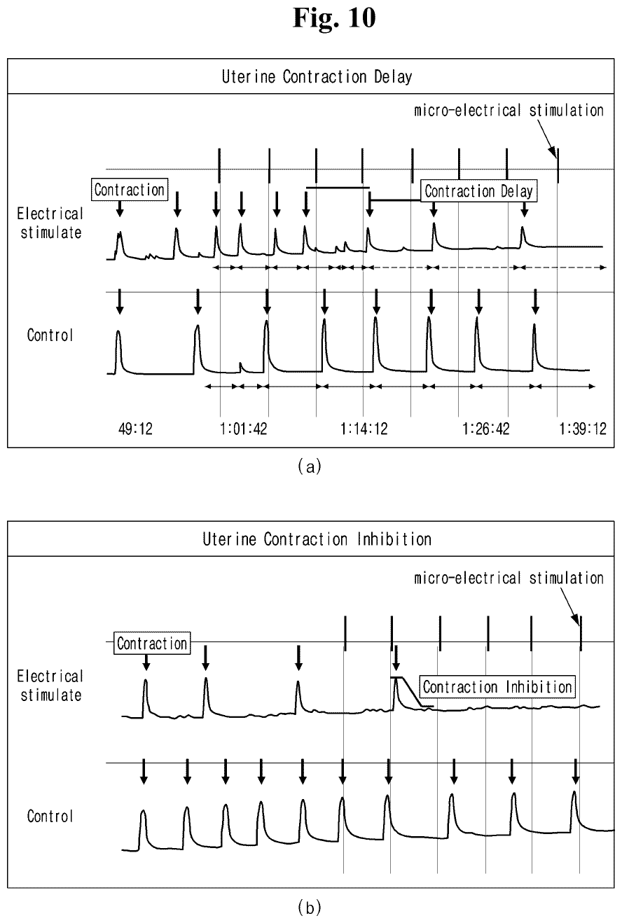

FIGS. 10 (a) and (b) are graphs showing uterine contraction delay and inhibition according to the intensity, interval, and duration of micro-electrical stimulation applied to human uterine smooth muscle.

Referring to FIG. 10 (a), when micro-electrical stimulation was applied to human uterine smooth muscle with an intensity of 200 μA, an interval of 200 μsec, and a duration of 20 seconds, it was confirmed that uterine contraction, which occurs once every 2.5 minutes, was delayed to 7.5 minutes. Referring to FIG. 10 (b), when micro-electrical stimulation was applied to human uterine smooth muscle with an intensity of 100 μA, an interval of 200 μsec, and a duration of 20 seconds, it was confirmed that uterine contraction was inhibited.

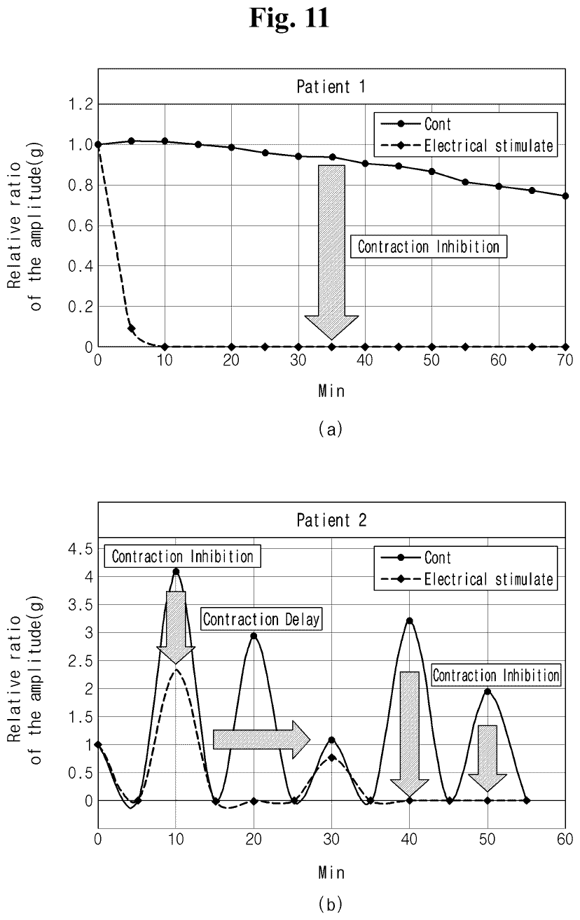

FIGS. 11 (a) and (b) are graphs showing uterine contraction delay and inhibition according to the intensity, interval, and duration of micro-electrical stimulation.

Referring to FIG. 11 (a), when micro-electrical stimulation was applied to human uterine smooth muscle with an intensity of 100 μA, an interval of 200 μsec, and a duration of 20 seconds, it was confirmed that uterine contraction was inhibited. Referring to FIG. 11 (b), when micro-electrical stimulation was applied to human uterine smooth muscle with an intensity of 400 μA, an interval of 200 μsec, and a duration of 20 seconds, uterine contraction was delayed, and when micro-electrical stimulation was subsequently applied with an intensity of 1000 μA, an interval of 200 μsec, and a duration of 20 seconds, it was confirmed that uterine contraction was inhibited.

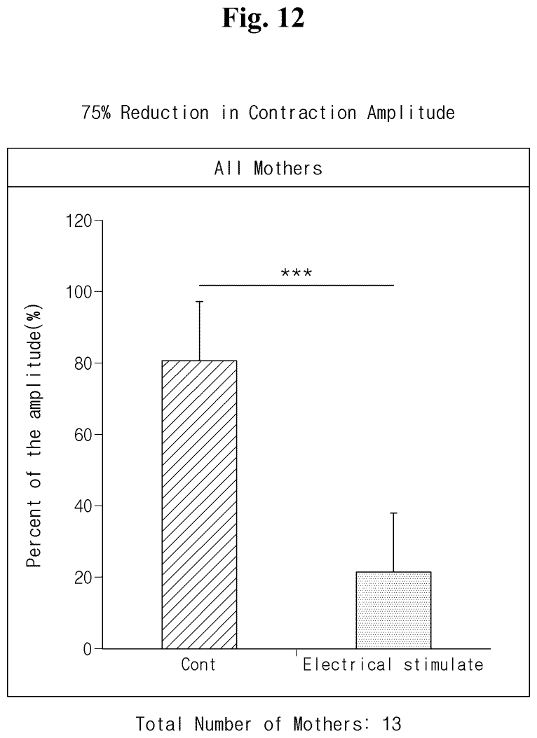

FIG. 12 is a graph confirming that uterine contraction decreased at a certain range of micro-electrical stimulation intensity for a total of 13 mothers.

Referring to FIG. 12, as a result of checking the uterine contraction amplitude, it was confirmed that uterine contraction was inhibited at micro-electrical stimulation intensities of 100 to 5000 μA. For all mothers, uterine contraction amplitude decreased by 75%.

Embodiments according to this specification can be implemented by various means, for example, hardware, firmware, software, or combinations thereof. In the case of hardware implementation, an embodiment of this specification can be implemented by one or more ASICs (application specific integrated circuits), DSPs (digital signal processors), DSPDs (digital signal processing devices), PLDs (programmable logic devices), FPGAs (field programmable gate arrays), processors, controllers, micro-controllers, micro processors, etc. In the case of firmware or software implementation, an embodiment of this specification can be implemented in the form of modules, procedures, functions, etc. that perform the capabilities or operations described above. Software code can be stored in the memory and driven by the processor. The memory can be located inside or outside the processor and can exchange data with the processor through various already known means.

On the other hand, it is possible to implement the embodiments of this specification as computer-readable code on a computer-readable recording medium. The computer-readable recording medium includes all types of recording devices in which data that can be read by a computer system is stored. Examples of the computer-readable recording medium include ROM, RAM, CD-ROM, magnetic tapes, floppy disks, optical data storage devices, etc. In addition, the computer-readable recording medium can be distributed to computer systems connected through a network, and computer-readable code can be stored and executed in a distributed manner. And functional programs, codes, and code segments for implementing the embodiments can be easily inferred by programmers in the technical field to which this specification belongs.

In the above, this specification has been examined focusing on its various embodiments. Those skilled in the art to which this specification belongs will understand that various embodiments can be implemented in a modified form without departing from the essential characteristics of this specification. Therefore, the disclosed embodiments should be considered not from a limiting perspective but from an explanatory perspective. The scope of this specification is not shown in the above description but in the claims, and all differences within an equivalent range should be interpreted as being included in this specification.

### INDUSTRIAL APPLICABILITY

The embodiments of this specification can detect the physiological phenomenon of uterine muscle contraction that appears in the induction mechanism of preterm labor to predict or continuously monitor the signs of preterm labor, and when uterine contraction is anticipated based on the detected neural signals, the uterine muscle contraction can be inhibited or delayed by automatically applying electrical stimulation to the uterine inlet nerve using a non-invasive neural electrode, thereby detecting preterm labor in advance.

1. A micro-electrical stimulation system for regulating uterine contraction, comprising:
a body that is non-invasively inserted into the uterus;
a stimulation neural electrode that inhibits uterine contraction by applying micro-electrical stimulation to the uterine inlet nerve; and
a controller that sets the intensity, interval, and duration of the micro-electrical stimulation,
wherein the controller sets the micro-electrical stimulation within an intensity of 100 to 5000 μA, an interval of 100 to 200 μsec, and a duration of 3 to 20 seconds.

2. The micro-electrical stimulation system for regulating uterine contraction according to claim 1, further comprising:
a recording neural electrode that measures neural signal information according to uterine contraction; and
a database unit that receives and stores the neural signal information from the recording neural electrode,
wherein the controller selectively forms a uterine contraction delay and inhibition state by applying at least two different micro-electrical stimulations based on the stored neural signal information.

3. The micro-electrical stimulation system for regulating uterine contraction according to claim 2,
wherein the controller:
acquires individual uterine contraction delay and inhibition information through changes in the neural signal information according to the intensity of the applied micro-electrical stimulation, and
determines the intensity, interval, and duration values of each micro-electrical stimulation based on the individual uterine contraction delay and inhibition information.

4. A method for controlling a micro-electrical stimulation for regulating uterine contraction, comprising:
(a) setting the micro-electrical stimulation within an intensity of 100 to 5000 μA, an interval of 100 to 200 μsec, and a duration of 3 to 20 seconds; and
(b) applying the set micro-electrical stimulation to the uterine inlet nerve by a stimulation neural electrode.

5. The method for controlling the micro-electrical stimulation according to claim 4, further comprising after step (b):
(c) measuring neural signal information according to uterine contraction by a recording neural electrode;
(d) receiving and storing the neural signal information from the recording neural electrode; and
(e) selectively forming a uterine contraction delay and inhibition state by applying at least two different micro-electrical stimulations based on the stored neural signal information.

6. The method for controlling the micro-electrical stimulation according to claim 5, wherein step (e) comprises:
(e-1) acquiring individual uterine contraction delay and inhibition information through changes in the neural signal information according to the intensity of the applied micro-electrical stimulation; and
(e-2) determining the intensity, interval, and duration values of each micro-electrical stimulation based on the individual uterine contraction delay and inhibition information.
